(19) United States
(12) Patent Application Publication
(10) Pub. No.: US 2021/0393957 A1
(43) Pub. Date: Dec. 23, 2021

(54) Systems and Methods for Sensing and Correcting Electrical Activity of Nerve Tissue

(71) Applicant: Tufts Medical Center, Inc., Boston, MA (US)

(72) Inventors: Pedram Hamrah, Wellesley, MA (US); Anam Akhlaq, Brookline, MA (US)

(21) Appl. No.: 17/281,430

(22) PCT Filed: Oct. 1, 2019
(86) PCT No.: PCT/US2019/054097

§ 371 Date: Mar. 30, 2021

Related U.S. Application Data
(60) Provisional application No. 62/740,202, filed on Oct. 2, 2018.

(51) Int. Cl. A61N 1/36; A61N 1/04; G02C 7/04

(52) U.S. Cl. CPC A61N 1/36046; A61N 1/0456; G02C 7/04; A61N 1/36031; A61N 1/36021

(57) ABSTRACT

Disclosed are apparatus, systems, devices, methods, and other implementations, including an apparatus that includes at least one contact lens fittable on an eye of a patient, with the contact lens including circuitry for receiving electrical activity signals associated with electrical activity produced by nerve tissue located proximal to the contact lens. The apparatus further includes a first sensor configured to sense the electrical activity produced by the nerve tissue and to provide the electrical activity signals, and a first stimulator to trigger a response in a body of the patient based, at least in part, on the electrical activity signals provided by the first sensor.

### CROSS-REFERENCE TO RELATED APPLICATIONS

This application claims the benefit of U.S. Provisional Application No. 62/740,202, filed Oct. 2, 2018, the contents of which are incorporated by reference.

### BACKGROUND

The cornea is the most densely innervated tissue in the body with around 300-600 times more nociceptors than the dermis and around 80 times more than the dental pulp. Immune function of the cornea is achieved by not only its role as a mechanical barrier, but also by the presence and interaction of immune cells, vessels and nerves. The sensory and motor components of these nerves play a role in perception of temperature, touch, pain and pressure as well as in blink reflex and tear production.

The short and long ciliary nerves of the ophthalmic branch of the trigeminal nerve enter the eye posterior to the globe. These nerves traverse above the choroid to reach the corneo-limbal junction. From there myelinated fibers traverse radially from the periphery to the central cornea where they lose the myelin sheet after approximately 2 mm and then move anteriorly to innervate the stroma and subsequently form the dense subbasal plexus below the corneal epithelium. Both unmyelinated and myelinated Mδ fibers (myelinated, fast conduction velocity) and C fibers (unmyelinated, slow conduction velocity) are involved in carrying pain sensation from the cornea.

Animal studies have been used to demonstrate corneal electrophysiology and to study the changes associated with various clinical diseases. Transmission of sensory input requires reception of signals by nociceptors and propagation of action potentials by the nerves. The cornea has several different types of receptors including thermoreceptors (10%), mechanoreceptors (20%) and polymodal (70%) receptors. Mechanoreceptors respond to mechanical stimuli phasically; while polymodal receptors, as the name implies, respond to heat, hyperosmolar solutions, mechanical force and chemicals, as well as, exogenous irritants and endogenous inflammation. Cold-sensitive thermoreceptors respond to cold and can be divided into two types: high-background, low threshold type and low-background, high threshold type. Mechanoreceptors have been shown to be involved in the detection of irritation and touch, while polymodal receptors have a definitive role in recognition of discomfort and pain, while their role in detection of itch is unclear. High-background, low threshold subset of cold receptors is implicated in dryness, while the low-background, high threshold subset is established in detection of coolness and pain.

In human subjects, pain and discomfort may be manifested as electrical activity produced by one or more nerves, such as the corneal nerves. Particularly, pain generation and transmission through corneal nerves is achieved by change in action potential due to ion movement across the nerves. Detection of stimulus by nociceptors generates a potential that is propagated along the nerves by an interplay of various ions. The neuron has a negative transmembrane resting potential due to high number of potassium ions inside the nerve cells, maintained by the action of Na+/K+ ATPase pump. With neuron stimulation, voltage-gated sodium channels open and a wave of depolarization is generated that travels along the neuron. At synapses, a change in voltage leads to neurotransmitter unloading into the synaptic cleft. This neurotransmitter binds to the ion channel and opens it, thus leading to ion influx and propagation of action potential across the synapses.

A patient's pain and discomfort can be the result of various underlying causes and conditions. For example, a patient wearing a contact lens may experience discomfort or irritation caused by the lens, with that discomfort or irritation manifested as a resultant electrical activity produced, for example, by the ophthalmic nerve. Similarly, various medical conditions or ailments the patient may be suffering from may result in respective electrical activities at one or more nerves (i.e., “nerve firing”) associated with those medical conditions or ailments.

### SUMMARY

Disclosed are apparatus, devices, systems, methods, and other implementations for a feedback system that provides stimulation to areas of a patient's body responsive to electrical activity (and/or other sensed activity) produced by nerve tissue (e.g., provide electrical stimulation to various nerves in the patient's body, provide chemical stimulation released from a controllable device holding a chemical or pharmacological agent, etc.) The applied stimulation may be used to correct abnormal electrical activities produced by nerve tissue. The electrical activity of the nerves is measured through sensors (e.g., electrodes), and provided to a lens worn by the patient that includes circuitry to interface with the sensors and transmitters (stimulators). In some variations, abnormality in the measured electrical activity may be detected through comparison of the electrical activity to baseline profiles representative of electrical activity in healthy subjects, and/or baseline electrical profiles representative of subjects suffering from different types of conditions or diseases (thus allowing more particular identification of the condition/disease afflicting the patient).

In some variations, an apparatus is provided that includes at least one contact lens fittable on an eye of a patient, with the contact lens including circuitry for receiving electrical activity signals associated with electrical activity produced by nerve tissue located proximal to the contact lens. The apparatus further includes a first sensor configured to sense the electrical activity produced by the nerve tissue and to provide the electrical activity signals, and a first stimulator to trigger a response in a body of the patient based, at least in part, on the electrical activity signals provided by the first sensor.

Embodiments of the apparatus may include at least some of the features described in the present disclosure, including one or more of the following features.

The contact lens may include the first sensor.

The first stimulator may be configured to produce electrical stimulation signals directed at tissue proximate to a location of the first stimulator.

The first stimulator may be configured to produce the electrical stimulation signals responsive to a determination that the electrical activity signals are abnormal. The electrical stimulation signals may be configured to correct, at least in part, the electrical activity produced by the nerve tissue.

The first stimulator may be configured to produce the electrical stimulation signals directed at one or more nerves in a body of the patient. The first stimulator configured to produce the electrical stimulation signals directed at the one or more nerves may be configured to produce the electrical stimulation signals directed at ophthalmic nerve tissue, including one or more of, for example, an ophthalmic nerve, branches of the ophthalmic nerve, or related parts of the ophthalmic nerve. The related parts may include cell bodies and/or synapses associated with nerve branch pathways.

The apparatus may further include a controller configured to determine whether the electrical activity signals are abnormal, and in response to a determination that the electrical activity signals are abnormal, generate modulating control signals to modulate electrical stimulation signals producible by the first stimulator, with the generated electrical stimulation signals applied to one or more tissue areas of the patient to reduce or impede abnormal electrical activity behavior produced by the nerve tissue.

The electrical activity signals may be representative of measured electrical activity waveforms generated due to nerve firing by at least one nerve. The controller configured to determine whether the electrical activity signals are abnormal may be configured to compare the measured electrical activity waveforms to a pre-stored baseline data representative of electrical activity waveforms. The controller configured to generate the modulating control signals may be configured to generate the modulating control signals that cause the first stimulator to generate modulating electrical stimulation signals applied to the one or more tissue areas to cause the at least one nerve or related parts of the at least one nerve to vary resultant electrical activity waveforms such that differences between the resultant electrical activity waveforms and at least one baseline waveform is reduced.

The controller configured to generate the modulating control signals may be configured to continually vary the generated modulating control signals responsive to variations in the measured electrical activity waveforms resulting from earlier modulating control signals.

The controller may further be configured to determine abnormality in electrical activity waveforms associated with patient pain or discomfort resulting from one or more of, for example, stimuli and conditions detected by thermoreceptors, stimuli and conditions detected by mechanoreceptors, and/or stimuli and conditions detected by polymodal and other nociceptors.

The contact lens may further include at least one of, for example, the controller, the first sensor, and/or the first stimulator.

The circuitry may further include the controller.

The apparatus may include a first contact lens and a second contact lens, with the first contact lens being couplable to the first sensor, and with the second contact lens being couplable to the first stimulator.

The apparatus may further include a first contact lens couplable to at least one first sensor and at least one first stimulator, and a second contact lens couplable to at least one second sensor and at least one second stimulator, with each of the first contact lens and the second contact lens being configured to alternately sense electrical activity of a respective at least one nerve and to stimulate respective tissue.

The first stimulator may include one or more stimulators that each produces one or more of, for example, electrical output, chemical output, mechanical output, thermal output, vibratory/tactile output, magnetic output, and/or optical output.

The first sensor may include multiple sensors, and at least one of the multiple sensors may be configured to sense the electrical activity produced by nerve tissue, and another at least one of the multiple sensors may be configured to sense at least one of, for example, chemical stimuli produced by the patient, mechanical stimuli, thermal, magnetic stimuli, and/or optical stimuli.

The first sensor configured to sense electrical activity produced by nerve tissue may further be configured to sense at least one of, for example, chemical stimuli produced by the patient, mechanical stimuli, thermal stimuli, magnetic stimuli, and/or optical stimuli.

The at least one contact lens may further be configured to correct vision attributes of the eye of the patient.

The first stimulator may further be configured to perform one or more of, for example, promote tissue growth, promote blood vessel growth, and/or trigger an immune system of the patient to counter a medical condition detected based, at least in part, on the sensed electrical activity produced by the nerve tissue.

The first stimulator may include an implantable device with a reservoir of chemical compound, the implantable device configured to controllably release the chemical compound in the reservoir based, at least in part, on the sensed electrical activity or other measured activity produced by the nerve tissue.

The circuitry may include a communication module, the communication module configured to communicate with one or more of the first sensor or the first stimulator via, for example, one or more wired connections, and/or one or more wireless connections.

The apparatus may further include a power source comprising one or more of, for example, a charging holding device including at least one of a battery or a capacitor, a mountable power source connectable to an external power supply, and/or a wireless power receiver module to generate electrical current from wireless transmissions received by the wireless power receiver module. The wireless transmissions may include one or more of, for example, RF transmissions, or optical radiation.

In some variations, a method is provided. The method includes establishing a communication link between circuitry, included in a contact lens fitted on an eye of a patient, and a first sensor configured to sense electrical activity produced by nerve tissue located proximate to the contact lens. The method further includes receiving from the first sensor electrical activity signals associated with the electrical activity produced by nerve tissue, and causing activation of a first stimulator to trigger a response in a body of the patient based, at least in part, on the electrical activity signals received from the first sensor.

Embodiments of the method may include at least some of the features described in the present disclosure, including at least some of the features described above in relation to the apparatus, as well as one or more of the following features.

Causing activation of the first stimulator to trigger the response in the body of the patient may include triggering electrical stimulation directed at one or more nerves in the body of the patient in response to a determination that the sensed electrical activity is abnormal.

The method may further include determining whether the electrical activity signals are abnormal, and in response to a determination that the electrical activity signals are abnormal, generating modulating control signals to modulate electrical stimulation signals producible by the first stimulator, the generated electrical stimulation signals applied to one or more tissue areas of the patient to reduce or impede abnormal electrical activity behavior produced by the nerve tissue.

The electrical activity signals may be representative of measured electrical activity waveforms generated due to nerve firing by at least one nerve. Determining whether the electrical activity signals are abnormal may include comparing the measured electrical activity waveforms to pre-stored baseline data representative of electrical activity waveforms, and generating the modulating control signals may include generating the modulating control signals that cause the first stimulator to generate modulating electrical stimulation signals applied to the one or more tissue areas to cause the at least one nerve or related parts of the at least one nerve to vary resultant electrical activity waveforms such that differences between the resultant electrical activity waveforms and at least one baseline waveform is reduced.

The pre-stored baseline data representative of the electrical activity waveforms may include one or more of, for example, a normal electrical activity waveform for a particular nerve, and/or a disease-caused electrical activity waveform for the particular nerve when a person is suffering from a particular irregular medical condition.

Generating the modulating control signals may include continually varying the generated modulating control signals responsive to variations in the measured electrical activity waveforms resulting from earlier modulating control signals.

The method may further include determining a medical condition that the patient is suffering from based on the sensed electrical activity produced by the nerve tissue.

The method may further include determining one or more of, for example, severity of the medical condition, and/or treatment and prognosis of the medical condition.

The method may further include generating storable electrical energy from wireless transmissions received by a power unit included with the circuitry of the contact lens.

Causing activation of the first stimulator to trigger the response based, at least in part, on the electrical activity signals may implement a biofeedback loop.

In some variation, a device is provided that includes a contact lens fittable on an eye of a patient, and circuitry included with the contact lens. The circuitry is configured to establish a communication link between the circuitry and a first sensor configured to sense electrical activity produced by nerve tissue located proximate to the contact lens, receive from the first sensor electrical activity signals associated with the electrical activity produced by nerve tissue, and cause activation of a first stimulator to trigger a response in a body of the patient based, at least in part, on the electrical activity signals received from the first sensor.

Embodiments of the device may include at least some of the features described in the present disclosure, including at least some of the features described above in relation to the apparatus and the method, as well as one or more of the following features.

The device may further include one or more of, for example, the first sensor, and/or the first stimulator.

Other features and advantages of the invention are apparent from the following description, and from the claims.

### BRIEF DESCRIPTION OF THE DRAWINGS

These and other aspects will now be described in detail with reference to the following drawings.

FIG. 1 is a diagram of an example system implementing a feedback mechanism to apply stimulation to a patient in response to sensed electrical activity produced by one or more nerves.

FIG. 2 is a block diagram of an example system, realizing a feedback model, for sensing electrical activity by nerve tissue and controllably applying stimulation in response thereto.

FIG. 3 is a schematic diagram of an example device which may be used to implement some of the various components and circuitries depicted in FIGS. 1 and 2.

FIG. 4 is a flowchart of an example procedure to perform stimulation of target tissue based on electrical activity produced by one or more nerves.

FIG. 5 is a graph showing recordings of nerve terminal impulse (NTI) activity measured for a mouse's eye nerves in response to different stimuli.

Like reference symbols in the various drawings indicate like elements.

### DESCRIPTION

Disclosed are devices, apparatus, methods and other implementations, to study and/or measure corneal nerve function and simultaneously or separately modulate stimulation applied to tissue (e.g., corneal nerves) to treat, for example, corneal nerve dysfunction in patients with corneal pain. Some of the examples implementations described in the present disclosure may be used to treat or alleviate symptom in such diseases and conditions that include, but are not limited to: 1) dry eye disease (DED), 2) neuropathic corneal pain (NCP) (including etiology related to post-surgical NCP, e.g. post-cataract surgery, post-LASIK surgery), psychiatric disease, neurological disease, autoimmune disease etc., 3) contact lens discomfort, 4) contact lens intolerance, 5) herpetic keratitis, 6) shingles, 7) other ocular diseases such as allergic keratitis, open-angle glaucoma, atopic keratoconjuctivitis, Fuchs' dystrophy etc., 8) diabetic neuropathy, and/or 9) other systemic diseases and conditions, including multiple sclerosis, fibromyalgia, migraines, Parkinson's disease, progressive supranuclear palsy, Crohn's disease, Fabry's disease, multiple endocrine neoplasia 2B. Other diseases and conditions not specifically mentioned herein may nevertheless also be treated by some of the implementations described in the present disclosure. The various diseases and conditions can induce anatomical and/or physiological changes in corneal nerves leading to chronic corneal pain and/or discomfort, or alternatively to corneal anesthesia. Anatomical changes may be seen as loss of nerves, increased tortuosity, decreased nerve density, and increased beading and nerve reflectivity as well as presence of neuromas on in vivo confocal microscopy. Physiological changes may be seen as modifications in nociceptor activity and a change in nerve electrophysiological firing patterns. Since the cornea is a highly innervated tissue with accessible location of sensory nerves, the solutions, approaches, and implementations discussed herein measure electrical signals from the nerves, and in response to the measured electrical signals (representative of electrical activity by the nerves resulting from possible conditions afflicting a patient), control/modulate stimulation applied to the body of the patient (e.g., in the form of electrical stimulation, also referred to as neuromodulation, applied to dysfunctional nerves) to alleviate pain or discomfort, or to achieve some other therapeutic objective. Accordingly, at least some of the implementations described herein may be used as a treatment modality for corneal pain.

Additionally, some of the apparatus, devices, methods and other implementations described herein may realize tools to measure and modulate corneal nerve electrophysiology to treat patients with dry eye disease, neuropathic corneal pain, post-herpetic neuralgia and systemic diseases affecting the cornea such as diabetes mellitus to study chronic corneal pain or nerve dysfunction and other ocular surface symptoms such as burning and discomfort. The quantification of electrophysiological activity through the implementations described herein could further be used to not only establish/improve the diagnostic criteria, but also to help compare efficacy of various treatment options, as well as find prognostic markers in patients with, for example, corneal pain.

In example embodiments described in the present disclosure, feedback mechanisms can be implemented to correct abnormal/aberrant electrical activity, produced by one or more nerves of a patient's body, through stimulation applied to the patient's body, be it electrical stimulation directed to nerve tissue of the patient, chemical stimulation in the form of controlled release of chemical agent at some selected location in the patient's body, thermal stimulation, optical stimulation (e.g., photo-induced stimulation), or mechanical stimulation such as vibratory stimulation. Measured electrical activity is provided to a lens worn by the patient. Sensors may be included with the lens (within the lens or disposed on a surface of the lens), but do not have to be in physical connection with the lens, and instead may simply be in electrical communication (realized through a wired or wireless link) with circuitry included with the lens. The stimulation (e.g., extent and type) that is responsive to the measured electrical activity can be controlled by the lens (e.g., through a controller or processor implementation that may be part of the lens' circuitry), or alternatively may be controlled by a device located remotely from the lens. In the latter situation, the circuitry of the lens may communicate data representative of the measured electrical activity (provided to the lens from the sensors) to a remote controller, and that remote controller may determine control signals, or actual stimulation signals, to control or regulate the stimulation that is to be applied to the patient. Determination of whether, and/or the extent and type of stimulation may be based on a determination of electrical activity abnormality. For example, measured electrical activity can be compared to baseline activity to assess the deviation of the measured activity from the baseline. The resultant stimulation control signal may be such that the resulting stimulation (e.g., electrical stimulation) causes reduction of the abnormality. Thus, the feedback mechanisms of some of the embodiments described herein can continually adjust the stimulation to be applied based on continually measured electrical activity of the nerves being monitored or sensed. Some conditions or ailments that may be treated using a lens-based feedback implementation include (as a representative, non-exhaustive examples), dry eye condition, post-herpetic neuralgia, and other diseases and conditions as discussed herein.

In some embodiments, the implementations may include an electrocorneogram (ECG) apparatus that can assess and quantify nerve (e.g., corneal nerve) function in real-time, based on which modulated stimulation can be used to treat pain or discomfort associated with the nerve function, or to correct the abnormality. For example, sensors associated with a contact lens can measure electrical activity of the corneal nerve indicative of pain or discomfort resulting from wearing the lens. If electrical activity indicative of pain is detected (because the data representative of the measured activity deviates from a pre-stored baseline data representative of normal activity, or matches baseline data representative of activity produced under various conditions or diseases), modulated (and adjustable) electrical stimulation directed at the nerve can be used to cause the electrical activity to be treated or otherwise restored to normal (thus alleviating the pain or discomfort that the patient/user is experiencing). Similarly, in other situations, involving other conditions, aliments, or other causes of discomfort and pain that are manifested as irregular/abnormal electrical activity produced by nerve tissue, controlled stimulation (electrical, mechanical, optical, or chemical) may be used to treat that condition or ailment by seeking to reduce and/or impede the irregularity of the measured electrical activity. Some of the embodiments described herein also include establishing normal waveforms activity (and/or waveforms produced by patients suffering from various ailments and conditions) to create a library of pre-stored (normative) data, so that data for a diseased state can be differentiated later on. Some of the implementations described herein can be thus be used to establish such nerve profiling patterns after stimulation of different nociceptors, so that the various nociceptors involved in different diseases may be distinguished.

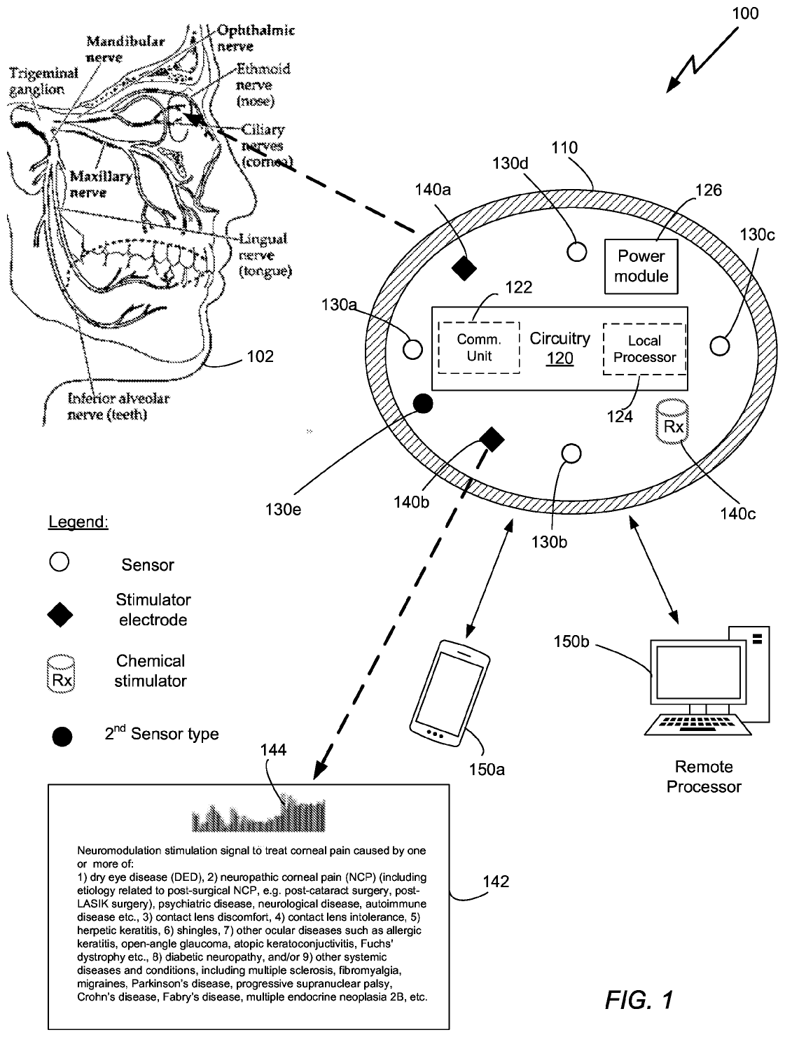

FIG. 1 is a diagram of an example system implementing a feedback mechanism, comprising a contact lens 110 fittable in an eye of a patient 102 and including circuitry to interface with one or more sensors. The feedback mechanism is configured to sense electrical activity of one or more nerves, and to apply stimulation to the patient in response to the sensed electrical activity produced by the one or more nerves (and optionally other activity). The various elements of FIG. 1 are not necessarily drawn to scale, but rather seek to illustrate the configurations and example implementations constituting an apparatus used to treat conditions that a patient is suffering from (including pain and discomfort that may have results from several conditions, including the mere wearing of a lens). While FIG. 1 depicts only a single lens, the user may be fitted with two lens, with one lens dedicated to sensing electrical activity (with that first lens thus constituting the electrocorneogram portion of the feedback system when the sensed nerves are the corneal nerves), and the other lens dedicated to control stimulation of nerve tissue responsive to the electrical activity detected by the first lens-based electrocorneogram implementation (the second lens thus constituting the neuromodulator portion of the feedback system). Alternatively, both lenses may each be used for sensing and stimulation, optionally at alternating intervals, e.g., one lens senses electrical activity, while the other lens performs stimulation of the patient's tissue. The lens 110 may optionally be a vision correcting lens, but in some embodiments, the lens 110 may not serve any optical function (such as correcting vision problems experienced by the patient 102).

As further shown in FIG. 1, the lens 110 includes circuitry 120 implementing interfacing and control functionality required to facilitate the sensing and/or processing of electrical activity by the nerve tissue being observed. The circuitry 120 may thus include, for example, a communication unit 122 configured to communicate, via wired or wireless links, with at least one of the sensors 130a-e of the system 100, and optionally communicate with one or more stimulators 140a-c. As will be discussed in greater detail below, the circuitry 120 may also include a local processor configured to determine whether (and/or the type, and to what extent) abnormality of electrical activity measured by the one or more sensors 130a-d exists (and/or whether abnormal activity measured by the sensor 130e, which may be a chemical sensor, or some other type of sensor, exists).

The contact lens 110 is structured to be placed on the cornea of the patient, and may include, possibly as part of the circuitry 120, a stimulator/transmitter (in a form of an integrated circuit, or chip) that can be embedded inside the lens or disposed on one of the surfaces of the lens to maintain close proximity to the corneal surface for adequate nerve stimulation (e.g., of corneal nerves). As will be discussed in greater detail below, in some embodiments, the lens may be developed as vision corrective or non-corrective lens, depending on the patient's needs. The possible types of materials that may be used to manufacture the lens 110 may include ionic or non-ionic, hydrophobic or hydrophilic materials. Examples of materials include hydrophilic acrylates, hydrophobic acrylates, rigid polymethyl methacrylate (PMMA), and polyurethanes, silicone hydrogels, silicone acrylates (SAs), fluoro-silicone acrylates, and various gas-permeable materials. The lens may also be hard lens, soft lens, or a hybrid lens comprising a soft clear center and a hard perimeter, that incorporates the stimulator (hereinafter, reference to “lens” refers to any type of lens, including any of a hard lens, a soft lens, or a hybrid lens). The stimulator/transmitter and electrodes may be embedded and placed inside the lens, so that they do not block the vision. The stimulator and electrodes may be placed in the concave surface of the lens, for maximum proximity to the ocular surface and best contact. However, some electrodes and stimulators/transmitters of the system 100 may be placed on the convex side, to make it less atraumatic for the corneal surface. In some embodiments, the implementation of the contact lens may be such that the weight of circuitry 120 chip may cause rotation of the lens; this may be needed to account for habituation. Alternatively, the circuitry 120 of the lens 110 may be of negligible weight or the weight of the simulator chip may be balanced by a counterweight to prevent lens rotation.

As noted, at least one of the sensors 130a-e is configured to sense the electrical activity produced by the nerve tissue (e.g., such as ophthalmic nerve). More particularly, the at least one of the sensors 130a-e is configured, in some embodiments, to measure activity by assessing nerve firing patterns. As noted, the contact lens 110 may include at least some of the sensors. For example, one or more of such sensors may be embedded within the lens structure and may constitute part of the circuitry 120 (alternatively, those sensors, while included with the lens 110, may nevertheless be a separate module or unit from the circuity 120 depicted in FIG. 1). Additionally or alternatively, one or more of the depicted sensors may be disposed on a surface of contact lens 110 (typically the concaved contact surface that contacts the cornea). It is to be noted that the sensors 130a-e are represented schematically in FIG. 1, and that the ring shapes representing the sensors 130a-d, or the circle shape representing the sensor 130e, do not necessarily require the sensors to have those shapes or structures. Rather, the sensors 130a-e may be of any appropriate shape or structure. For example, the electrode sensors 130a-d may be ring-shaped, rectangular, round, or otherwise shaped or structured to achieve some desired pre-determine electromagnetic or electrical sensory behavior. It is also to be noted that the circular or ring-shaped symbols could correspond to sensor devices with complex geometrical structures (e.g., to perform functions such as measuring intraocular pressure, measure chemical reactions, detect or sense optical radiation, etc.)

An advantage of having sensors that are embedded within the lens structure (as opposed to being disposed on the contact surface) is that the embedded sensors cause less discomfort to the user of the lens 110 (but at a cost of reduced measurement sensitivity). Some of the sensors of the system 100 may be placed within or at the tissue that is to be monitored, and thus will not be in direct physical contact with the lens 110. For example, the one or more sensors may be placed in proximity to the ophthalmic nerve illustrated in FIG. 1, while other sensors may be placed proximate other nerve tissue (e.g., at the skin surface locations that are proximate to the locations of the nerve(s) to be monitored). Such sensors may also be implanted within the body of the patient (including within the head section and/or other parts of the body), to be in close proximity to the target nerve tissue whose electrical activity is to be monitored, or may be placed on the skin surface of the patient, which avoid the invasiveness of implanting an internal sensor, but at the cost of reduced sensitivity to the electrical activity to be monitored. For those sensors (also referred to as a remote sensor) that are not in direct physical contact with the contact lens, electrical activity signals sensed or measured by such sensors are communicated to the circuitry 120 of the contact lens (which, as noted, may include the communication unit 122) via wireless or wired communication links

Measuring electrical activity of nerve tissue avoids more invasive procedures to determine the existence of various conditions or diseases. For example, corneal electrophysiology avoids the need to perform confocal microscopy procedures, and can provide a window into anatomical changes of nerves, allowing corneal electrical nerve function to be assessed and quantified in real-time (i.e., anatomical changes of the nerves can be inferred from the nerve function and pathophysiology). Thus, the sensing of electrical activity by at least some of the sensors of the system 100 facilitates detection and assessment of the symptoms of diseases/conditions the patient may be suffering from (e.g., detection of corneal diseases or conditions, including corneal pain, burning, etc.) The sensors' measurements of electrical activity produced by nerve tissue may, as will become apparent below, be compared to a baseline (of nerve firing profiles for healthy individuals as well as for individuals suffering from various diseases or conditions) established at an earlier time through a procedure involving controlled stimulation of the nerve tissue (e.g., stimulating the corneal nociceptors via various stimuli including cold, heat, chemical, pressure, light, and/or magnetic stimulation). As described herein, in addition to determining the appropriate stimulation responses to apply to a patient, measuring the electrical activity of nerve tissue may be used to establish/improve diagnostic criteria, establish association between symptom severity and clinical signs, assess responses to therapy and establish prognostic markers, and other applications.

The electrical activity produced by nerve tissue can be measured across nerves as electrical signal in several areas of the human body. For example, to measure electrical activity at a particular tissue area, electrodes can be placed at two points (as a recording electrode and a reference electrode) and the potential difference across these points can be measured. A ground electrode may also be used in such systems. The configuration and/or structure of one or more of the sensors used by the system 100 may thus be similar to sensor configuration used, for example, for producing an electroretinogram (ERG) for a retina (sensors configured specifically for measuring electrical activity in corneal nerves and other nerves may be used instead of, or in addition to, sensors that typically are used for ERG applications). An ERG device generates a waveform that represents electrical activity of the retina by mapping changes in ion movements at the level of photoreceptor layer in response to dark and light stimuli (an ERG device may use different types of electrodes, such as Burian-Allen electrodes, Dawson-Trick-Litzkow electrodes (DTLs), gold/copper wire electrodes, ERG-JET electrodes, Hawlina-Konec electrodes, cotton-wick electrodes and skin electrodes). Since pain signals are caused by changes in ion movement, and because corneal nerves are at an easily accessible location, a sensor configuration similar to that used for ERG may be used, for at least some of the sensors 130a-e, to assess corneal functional nerve responses that result in ocular symptoms. Example corneal electrodes to measure electrical activity associated with pain or discomfort signals from the cornea may include hard lens electrodes (electrodes embedded within, or disposed on the surface of, hard lenses), soft lens electrodes, or Duette hybrid lens electrode.

In some embodiments, at least one of multiple sensors (e.g., such as any of the sensors 130a-e) used with the apparatus implementing the electrical-activity-measurement and stimulation feedback mechanism of the system 100 may be configured to sense the electrical activity produced by nerve tissue, while another at least one of such multiple sensors (such as the sensor 130e, but also any of the other sensors) may be configured to sense at least one of, for example, chemical reactions (e.g., chemical reactions produced by the patient), reactions responsive to mechanical stimuli, reactions to optical stimuli, etc. In such embodiments, generating controlled/modulated stimulation (e.g., to relieve symptoms, counter abnormal electrical activity by nerves to correct their behavior) may thus be based on various types of sensed reactions, rather than just sensed electrical activity produced by one or more nerves. In some embodiments, a particular sensor (such as any of the sensors 130a-e) may be configured to sense more than one type of stimulus, e.g., to sense electrical activity as well as mechanical, chemical, optical, and/or any other type of activity.

For example, the system 100 may include one or more types of biosensors (included within the lens 110, e.g., embedded in the lens body or disposed on the lens' surface, or located remotely from the lens) such as an intraocular pressure sensor configured to detect fluctuations in corneal curvature associated with changes in pressure, a light sensor that can gauge the level of oxygenation and pulse rate from conjunctival blood vessels by using photodiodes that emit light and measure the amount of light transmitted through the vessel, a sensor to measure one or more environmental conditions of the area where the patient is located (e.g., to measure light intensity and humidity), a sensor configured to analyze substrates in tears (including blood glucose levels), etc. Additional examples of sensors that may be included with the system 100 (to provide the input to the stimulation-based feedback mechanism implementation of the system 100, or to capture supplemental data that may be used for other purposes) may include a magnetic and/or video-based sensor (e.g., included in the lens 110) to track eye gazing and blinking in order to assess factors such as the psychological state of the user. In another example, a video sensor (also included in the lens) may be used to implement motion capture functionality (e.g., by tracking eye movements and recording the positions, angles, velocities and impulses, and accounting for overlap by using three-dimensional representation). Pupil sensors in the contact lenses may to be incorporated in ‘smart’ contact lenses to assess responses such as dilation. Pupil tracking and diameter change may also be used in applications such as tracking/assessing mental context (a ‘smart’ contact lens may also be used in the development of camera, that can take pictures with blinking and can accommodate with pupillary responses).

With continued reference to FIG. 1, the system 100 includes one or more stimulators 140a-c. In the example of FIG. 1, two electrode stimulators (represented graphically as diamonds) are included with the lens 110 (e.g., either embedded within the lens' body, or disposed on the convex or concave surfaces of the lens 110). The system 100 may also include one or more remote electrode stimulators, e.g., implantable electrode stimulators, or stimulator device(s) that are securable to the patient's body. In addition the system 100 also include, the example embodiments of FIG. 1, at least one chemical stimulator 140c (which may be included with the lens 110, or may be implantable or securable to the body of the patient) and which typically contains some chemical substance or agent (e.g., a pharmacological substance) that can be controllably released in response to a control signal received by the stimulator 140c. It is to be noted that the stimulators are represented schematically in FIG. 1, and that the diamond shape representing the stimulators 140a-b do not necessarily requires those stimulators to have those shapes or structures. Rather, the electrode type stimulators 140a-b may be of any appropriate shape or structure. For example, the electrode-type stimulators may be rectangular, round, ring-shaped, or otherwise shaped or structured to achieve some desired pre-determine electromagnetic or electrical stimulation behavior.

In some embodiments, at least one of the stimulators (i.e., a first stimulator of the multiple deployed stimulators) may be configured to produce electrical stimulation signals directed at tissue proximate to a location of the first stimulator. For example, as depicted within inset 142 in FIG. 1, an example stimulation signal 144 may be applied by the stimulator 140b in response to sensed electrical activity produced by a nerve tissue (with the stimulation signal 144 produced upon a determination that the sensed electrical activity is abnormal as compared to a baseline of normal electrical activity produced by the particular nerve tissue). As noted, the stimulation signal (be it an electrical signal, such as the signal 144, or some other type of stimulus) may be configured to treat (e.g., alleviate) symptoms such as pain (manifested, in part, by abnormal electrical activity by a particular nerve tissue such as the corneal nerve), caused as a result of one or more medical conditions/diseases. Such medical conditions/diseases may include (but are not limited to) one or more of: 1) dry eye disease (DED), 2) neuropathic corneal pain (NCP) (including etiology related to post-surgical NCP, e.g. post-cataract surgery, post-LASIK surgery), psychiatric disease, neurological disease, autoimmune disease etc., 3) contact lens discomfort, 4) contact lens intolerance, 5) herpetic keratitis, 6) shingles, 7) other ocular diseases such as allergic keratitis, open-angle glaucoma, atopic keratoconjuctivitis, Fuchs' dystrophy etc., 8) diabetic neuropathy, and/or 9) other systemic diseases and conditions, including multiple sclerosis, fibromyalgia, migraines, Parkinson's disease, progressive supranuclear palsy, Crohn's disease, Fabry's disease, multiple endocrine neoplasia 2B, etc.

The electrical stimulation signals can be generated using power delivered from a local power source electrically coupled to the first stimulator (e.g., a power source included with the lens 110 in circumstances where the first stimulator is electrically coupled to the lens through a wired connection). In some situations, the first stimulator (e.g., an electrode-based stimulator), may include circuitry, including a communication module and/or wireless power receiver, to harvest power from wireless power transmissions, and generate electrical stimulation directed to the tissue proximate to the location of the first stimulator.

In some embodiments, a first stimulator is configured to produce the electrical stimulation signals responsive to a determination that the electrical activity signals are abnormal, with the electrical stimulation signals being configured to correct, at least in part, the electrical activity produced by the nerve tissue (and measured by one or more of the sensors interfacing with the lens 110 of the system). For example, the first stimulator may be configured to produce the electrical stimulation signals directed at one or more nerves in a body of the patient, which may be at least some of the same one or more nerves whose electrical activity was measured by the one or more sensors (thus establishing the feedback functionality in which neuromodulation to stimulate particular nerves is performed in response to the electrical activity produced by those nerves). An example of a nerve that the first stimulator may neuromodulate is the ophthalmic nerve. In such embodiments, that stimulator may be configured to produce the electrical stimulation signals directed at the ophthalmic nerve and related parts of the ophthalmic nerve, including one or more of, for example, branches of the ophthalmic nerve, nociceptors of the ophthalmic nerve, cell body of the ophthalmic nerve, and synapses of ophthalmic nerve. It is to be noted that in some examples, a sensor device may also be used as a stimulator. For example, the sensor 130a may be configured to both measure electrical activity produced by nerves proximate to the location of the sensor 130a, and to also direct electrical stimulation signals at the nerve tissue proximate the sensor 130a.

As noted, at least one of the stimulators of the system 100 may be placed inside the circuitry 120 included in the lens, or may be placed in other locations in the ring structures of the lens (and the chip may serve as both stimulator and transmitter). Alternatively, the stimulator may be present externally and directly connected to the power source to serve as a separate unit. In the latter case, energy from the stimulator may then be transmitted wirelessly to the transmitter inside the lens (e.g., powering of the circuitry of the lens, and modules coupled thereto, can be achieved by wirelessly delivering some of the power directed from the power source to the stimulator). The stimulus may, in some variations, be electrical in nature, ensuring energy is transmitted to the nerves for neuromodulation. In cases where the stimulus is non-electrical in nature, the electrodes may be designed accordingly. For example, where the stimulus produced by the stimulator is mechanical, the stimulator may include a transducer to convert actuation/control signals to mechanical waves, possibly with the use of magnets, piezo-electric elements, etc. Such mechanical waves may be configured to, for example. stimulate the mechanoreceptors in the cornea. In case the stimulus is vibratory in nature, the electrodes may be able to generate ultrasonic energy by use of transducers or vibratory motors. Other examples of possible stimuli generated by the one or more stimulators of the system 100 include thermal stimuli, photo-induced stimuli, and magnetic stimuli. In case a chemical stimulus is needed, the contact lens may have reservoir/pits (schematically illustrated as the stimulator 140c) with an automated delivery system and a mechanism to refill the pit to replace the chemicals in the reservoir. In such cases, the part of lens containing the reservoirs may also be detachable/disposable. The chemicals that may be used comprise, but are not limited to, histaminic/nicotinic receptor agonists and irritants such as ammonia, benzene, nitrous oxide, capsaicin, mustard oil, horseradish, crystalline silica, etc. It should be noted that these same chemicals may be used for measuring the responses of nociceptors (e.g., to establish baseline data).

Electrical output may be delivered to the corneal nerves by electrodes that may be in contact with the cornea. These electrodes may be fixed to the contact lens in a ring or chip (i.e., the circuitry 120) by suitable anchorage such as biocompatible nails, glue etc. The electrodes provided directly on the lens (as part of an IC circuitry, or as separate units in electrical communication) are constructed from conductive materials. These conductive materials may include metals such as stainless steel, titanium, tantalum, platinum or platinum-iridium, other alloys, conductive ceramics such as titanium nitride, liquids, and gels. The electrodes may have any shape ranging from ellipsoid, spherical, ovoid or cylindrical (as noted, the graphical symbols used in FIG. 1 to designate the stimulators are schematic representations of the stimulators, and are not meant to represent any specific structure of the electrodes). The electrodes 140a-b may comprise materials that promote electrical contact, while providing an interface for conduction. For interface, hydrogel may be used to coat the end of electrodes to reduce impedance and to make the contact lenses relatively atraumatic for the eye. The contact lenses may also be impregnated with gel/fluid using a foam/porous material, to reduce impedance and to account for lack of tears/wet surface in dry eye disease.

The electrodes may stimulate both eyes substantially simultaneously. Alternatively, the electrodes may alternate to stimulate the eyes in a sequential fashion, so that the charges from both eyes do not cancel each other. In another embodiment, the electrode signal to one eye is able to affect/stimulate both the eyes. Alternatively, two electrodes (one electrode per eye) may be placed in a manner that one electrode may serve as transmitter and one as receiver (e.g., one electrode may trigger electrical stimulation by directing electric current at nerve tissue, while another electrode may be configured to sense electrical activity). The electrodes may be configured to have a constant or fixed function, or may alternate between their functions as transmitters and receivers. Insulation materials such as a flexible polymers (thermoplastic elastomer or alloys, thermoplastic polyurethanes etc.) or silicone may be used to insulate the lenses, with the exception of electrodes.

Neuromodulation signals produced, for electrode-type stimulators that direct electric current at target nerve tissue, may have various waveforms, to achieve general, patient-specific or etiology-specific neuromodulation in patients with NCP, DED, post-herpetic neuralgia and other diseases or conditions as discussed herein. This signal variety may be produced by signal modulation at the level of power source, stimulator chip or both. Photovoltaics may be used to manipulate the signal of the power source. For example, by using an optical modifier with the power source and a photodiode as a receiver, various intensity, frequency, timing and amplitude signals may be produced. Alternatively, the stimulator chip may receive an initial raw stimulation signal, and produce resultant output after modification for frequency, shape, pulse width and amplitude, while the original power source signal may be stable or only vary in amplitude and voltage. The generated stimulation waveforms (for neuromodulation functionality) may be continuous or pulsating, monophasic or biphasic. In case of continuous waveforms, the shape may range from sinusoidal, quasi-sinusoidal, square, saw-tooth, triangular, truncated to irregular forms. In case of pulsatile waveforms (i.e., on and off period)6, the inter-pulse interval may vary. Both pulsatile waveform and variations in inter-pulse intervals may reduce habituation. If the waveform is biphasic, it may be symmetric or asymmetric. If the waveform is monophasic, it may or may not need a charge-balancing phase. In biphasic waveforms, charge balancing may be ensured. The frequency, amplitude and pulse-width may remain the same from wave to wave or may vary. The amplitude and frequency of the signal may be of irregular pattern, in increments or in decrements to reduce habituation. The incremental pattern of amplitude may also help increase patient comfort. Additionally, in case of biphasic waveform, the phases may be either voltage-controlled or current-controlled. Alternatively, one phase of the biphasic pulse may be current controlled and one phase may be voltage controlled.

In some embodiments, the frequency used for the waveforms may range from 0.1 Hz to 200 Hz. Probable ranges of frequency that may most likely be used include 10-60 Hz, 25-35 Hz, 50-90 Hz, 65-75 Hz, 130-170 Hz, and 145-155 Hz. In case of current-controlled stimulus, the amplitude may range from 10 μA-100 mA, though most likely the device may have an amplitude between 0.1-10 mA. In case of voltage-controlled pulse, the amplitude may range from 10 mV-100 V, though it may most likely lie in the range of 5-50V. The pulse width may also vary between 1 μs-10 ms, although ranges such as 10-100 μs and 0.1-1 ms may be most likely used. Each pulse may be current-controlled or voltage-controlled, or consecutive pulses may be controlled by such that one pulse is voltage controlled and the next is current controlled or vice versa. In some variations, where the pulse waveform is charged-balanced, the waveform may comprise a passive charge-balancing phase after delivery of a pair of monophasic pulses, which may allow the waveform to compensate for charge differences between the pulses.

In some embodiments, the system 100 may store in a memory storage device (which may be part of a controller or processor, discussed in greater detail below) data representative of the most recent electrical waveform discharged by any one of its electrode-type stimulator. Such a memory storage device may also be used to store data representative of other types of controlled stimulations, such as mechanical, optical, or chemical stimulation triggered by non-electrode-type stimulator. For example, data representative of the most recent mechanical stimulation (e.g., vibrating transducers actuated through control signals provided to the stimulator), including the force and pattern of the vibration, the time at which the mechanical stimulation was applied, the identity of the stimulator that applied that stimulation energy, etc., may be recorded in the memory storage device. The memory storage device may be configured to store only the most recently applied stimulation signal by each of the system's stimulators, or to also record earlier stimulations. Thus, the system 100 has access to information for at least the most recent applications of stimulation by its stimulators (e.g., any of the stimulators 140a-c of FIG. 1) prior to cessation of the activity by any one of the stimulators. Hence, the same waveform (or some resultant waveform that is adjusted as a function of elapsed time since the last stimulation was applied) may be restarted when the device (or individual stimulators) switches on again. Alternatively, the data may be recorded but every time the device switches on, the signal may reboot and readjusted, according to the need of the user. Additionally, in some embodiments, both the eyes (and/or other parts/organs of the body) may be subjected to the same or different waveform, with, in the case of electrical stimulation applied to nerve tissue of the eyes, charge balancing phase to the contralateral eye. Alternatively, the stimulus to the eyes may have inter-pulse interval (i.e. input to one eye at a time), to reduce any cancellation effect from the contralateral eye.

As noted, the stimulators of the system 100 deliver controlled or modulated stimulation to trigger a response in the body. The nature of the controlled/modulated stimulation is determined by a controller 124 (e.g., a processor-based device) based on electrical activity signals provided by the sensors, e.g., communicated to the circuitry 120 included in the lens 110. Thus, in some embodiments, the system 100 further includes a controller configured to determine whether the electrical activity signals (provided by at least one of the sensors) are abnormal, and in response to a determination that the electrical activity signals are abnormal, generate modulating control signals to modulate electrical stimulation signals producible by the stimulator, with the generated electrical stimulation signals applied to one or more tissue areas of the patient to reduce and/or impede abnormal electrical activity behavior produced by the nerve tissue. In some examples, the controller may be also included in the lens, e.g., the controller may be part of the circuitry 120, or may be a separate module that is either embedded in the body of the lens or is disposed on one of the lens' surfaces. In such embodiments, the underlying data (e.g., at least the signals representative of the electrical activity produced by one or more nerves) based on which the stimulation signals are derived is processed locally at the lens, and control signal that control the generation of stimulation signal, or actual modulating stimulation signals, are provided to one or more of the stimulators of the system 100. Alternatively, at least some of the processing applied to the sensed data (which includes the electrical activity signals sensed by at least one of the system's sensors) may be assigned to a remote processor, such as the computing device 150a or 150b (which may be in wireless or wired communication with the circuitry 120 of the lens 110). The remote computing device 150a may be a mobile device (such as a smartphone), while the example remote computing device 150b may be a stationary node, such as a stationary computer terminal, an access point, etc. In embodiments in which a remote processor is used, that remote processor may perform the determination of whether the electrical activity signals produced by the sensors are abnormal, and generate control signals (to actuate stimulators so as to cause them to trigger appropriate stimulation signals) or actual stimulation signals that are sent to the respective stimulators of the system 100 (either directly to the stimulators when such stimulators include dedicated communication modules, or indirectly via an intermediary communication module implemented as part of the circuitry 120).

In some examples, the electrical activity signals are representative of measured electrical activity waveforms generated due to nerve firing by at least one nerve (such as the ophthalmic nerve, whose electrical activity may be sensed by one or more sensors). In such examples, the controller configured to determine whether the electrical activity signals are abnormal may be configured to compare the measured electrical activity waveforms to a pre-stored baseline data representative of normal electrical activity waveforms. The controller may also be configured, in such examples, to generate the modulating control signals that cause at least one stimulator to generate modulating electrical stimulation signals applied to the one or more tissue areas to cause the at least one nerve or related parts of the at least one nerve to vary resultant electrical activity waveforms such that differences between the resultant electrical activity waveforms and at least one baseline waveform (which may be representative of a normal electrical activity waveform) is reduced. In some examples, the pre-stored baseline data representative of the electrical activity waveforms may include one or more of, for example, a normal electrical activity waveform for a particular nerve, or a disease-caused electrical activity waveform for the particular nerve when the person is suffering from a particular medical condition (i.e., the expected waveform for a particular nerve that would be observed if the patient were suffering from that particular medical condition).

More particularly, in these examples, the system 100 may have access to a repository of pre-stored data representative of a baseline of electrical activity waveform from healthy individuals (the repository may be individualized for the specific patient with respect to whom the system 100 is to be used), and/or a baseline of waveforms corresponding to the electrical activity waveforms that would be produced when the corresponding individual suffers from various conditions or diseases. Such waveforms may be associated with pain or discomfort that are produced when a person is suffering from a particular ailment or condition, and therefore the particular electrical activity pattern that is sensed may be indicative of a particular condition or ailment. If it is determined that an abnormality exists in the received electrical activity waveforms (e.g., the deviation of a sensed electrical activity signal, as determined by comparisons of samples of the measured signals to one or more of the baseline signals, or as determined from comparison of signal characteristics of the sensed waveform and one or more of the baseline waveforms, exceeds some pre-determined threshold), a stimulation signal may be applied to one or more tissue areas of the patient to cause a reduction in the abnormality of subsequent measured electrical activity signals. For example, the generated stimulating signal may be an electrical activity signal applied to a nerve that, when added to the current electrical activity produced by the nerve, offsets or even cancels (e.g., through destructive interference) the electrical activity produced by the targeted nerve. In some embodiments, the stimulation generated (be it an electrical stimulation, mechanical stimulation, etc.) may be applied to some part of the body to result in a response that mitigates the aberrant electrical activity (e.g., causing the nerve producing the aberrant/abnormal activity to produce, in response to the stimulation, a modified electrical activity behavior).

In some examples, a baseline waveform(s) from the cornea for a particular individual (e.g., the patient on which the system 100 is to be used), produced by the corneal nociceptors, may be is recorded. In some embodiments, the corneal nociceptors can be stimulated via various stimuli, including cold, heat, chemical, pressure, and/or light. This will be used to establish a baseline and profile different firing patterns to assess the influence of those nociceptors (including polymodal, thermal and mechanical receptors) in ocular diseases associated with corneal pain, including DED, NCP and post-herpetic neuralgia, as well as systemic corneal neuropathies. This information can further be translated to target the specific nociceptors involved in each patient or disease.

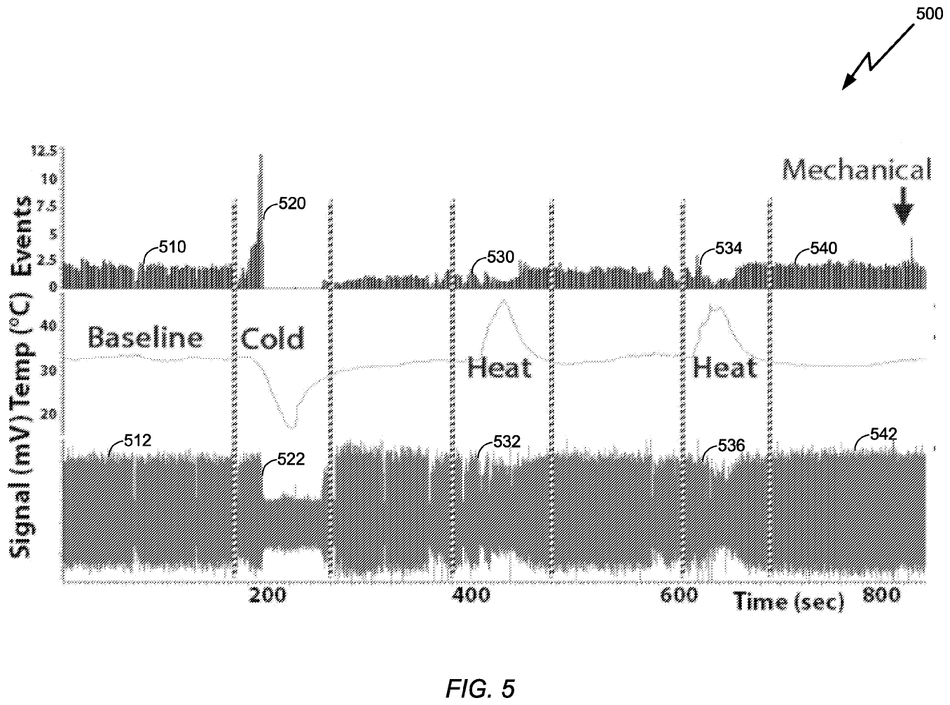

For example, with reference to FIG. 5, a graph 500 showing recordings of nerve terminal impulse (NTI) activity measured for a mouse's eye nerves in response to different stimuli, is provided. The graph 500 includes baseline responses 510 and 512, cold stimuli response 520 and 522 corresponding to the monitored nerves' electrical responses when the temperature affecting the eye nerves was lowered, heat stimuli responses 530, 532, 534, and 536 corresponding to the monitored nerves' electrical responses when the temperature was increased, and mechanical stimuli responses 540 and 542 corresponding to the monitored nerves' electrical responses when mechanical stimuli (suction applied to the corneal surface with a glass micropipette) was applied. Similar pre-recorded baselines responses can be obtained for prospective human patients for which the neuromodulation approach described herein (using a biofeedback loop in which stimuli is generated responsive to measured nerves' electrical activity) is to be applied.

Furthermore, nerve firing patterns and changes in thresholds may be observed and profiled for different diseases and conditions. This can be used to identify, based on sensed waveforms, whether a patient may be suffering from some particular condition or disease (e.g., using waveform analysis, a learning engine that receives the waveforms as input, etc.)

In various examples, the controller configured to generate the modulating control signals may be configured to continually vary the generated modulating control signals responsive to variations in the measured electrical activity waveforms resulting from earlier modulating control signals. That is, the controllable/modulated stimulation may be an iterative process by which, in response to a determination that sensed electrical activity produced by one or more nerves is abnormal, the controller generates modulating control signal that are provided to the one or more stimulators of the system 100 to trigger a stimulation action intended to reduce or impede the abnormality of the electrical activity. Having applied a stimulation action, the one or more sensors measuring the electrical activity of the nerve tissue sense the resultant electrical activity, which is provided to the controller to determine if the resultant activity is converging to a baseline waveform. If there is some convergence, the controller may continue to determine appropriate modulating control signals to continue applying stimulation action. This process can continue until (or even subsequent to) the abnormality being eliminated. In situations where the controller determines a control signal, or a stimulation signal, that causes a worsening of the abnormality in the electrical activity, the controller may, in response to that determination, derive an adjustment to the control signal or stimulation signal. For example, the controller may reverse the direction at which parameters controlling the signal(s) generated are being adjusted (e.g., decreasing the value(s) of one of the parameters, such as a voltage, phase, or frequency, controlling the characteristics of the generated signal if in the previous iteration that parameter's value was increased and as a result the abnormality between the measured electrical activity and the baseline worsened).

Turning back to FIG. 1, the lens 110 may also include a power module 126. In some embodiments, the power module may comprise a lithium battery or rechargeable batteries. Alternatively, a mountable power source may be developed to charge the stimulator. In that case, the wires used in the power module may comprise one or more conductive materials such as stainless steel, titanium, platinum or platinum-iridium, other alloys or titanium nitride etc. In some embodiments, wireless energy transmissions by laser diode or light-emitting diode (LED), that may or may not use infra-red light, may be used. Infrared light may be used in wavelength spectrum of 880 nm and 930 nm, as it is not perceived with the human eye, yet can be detected by silicon-based photodiodes. Another optional embodiment includes use of an optical modifier, with or without condenser lens and microlens array, to produce light in various frequency spectrums. The wavelengths used would comply with American National Standard Institute (ANSI) standards, to ensure retinal safety. An on/off switch may be included so that the stimulator may be switched off when not in use by patient. Another possible implementation is the use of electromagnetic modifiers, instead of optical modifiers, as an energy source and related equipment for receiver. Thus, the system 100 may include, in some embodiments, a power source comprising one or more of, for example, a charge holding device such as a battery or a capacitor, a mountable power source connectable (e.g., via an interfacing port) to an external power supply, or a wireless power receiving unit (i.e., a wireless power harvesting unit) configured to generate electrical current from wireless transmissions received by the wireless power receiver module, with such wireless transmissions including one or more of RF transmissions, or optical radiation (e.g., optical radiation in the visible range, infrared radiation, etc.)

User control of the system 100 may be effectuated through a user interface provided, for example, through a remote device that can be used to adjust operation of the sensors, the lens circuitry, the stimulators, and/or the controller (analyzing the data collected via the sensors and generating stimulation signals or modulating control signals to trigger stimulation signals). The user may be able to adjust the input from the lens by using the output interface. The user interface may be present as a unit of the external power source, or a software application running on a remote processor device such as the remote mobile device 150a or the remote computing device 150b. Using an on/off control on the user interface, the various units of the system 100 may be individually or collectively powered on or off The interface may also comprise several other controls (buttons/knobs/levers/sliders/touchpad) to change settings of frequency, intensity, pattern, and time duration for stimulation signals. The user interface may also include a screen/display to view the sensed waveforms provided by one or more of the sensors of the system 100, and have indicators for the user to show status of, or changes to, the system's settings, with such indicators implemented using light, sound, vibration, tactile clicks, etc. For example, a green light may be activated to indicate when the particular units of the system are charged. In embodiments in which a screen or a display device are used as part of the user interface, numerical data may be displayed for the various system settings. The interface may also be controlled using voice command. This may be helpful in highly photophobic or legally blind patients. The user interface may also include memory storage devices to record and store data received or generated by the user interface.

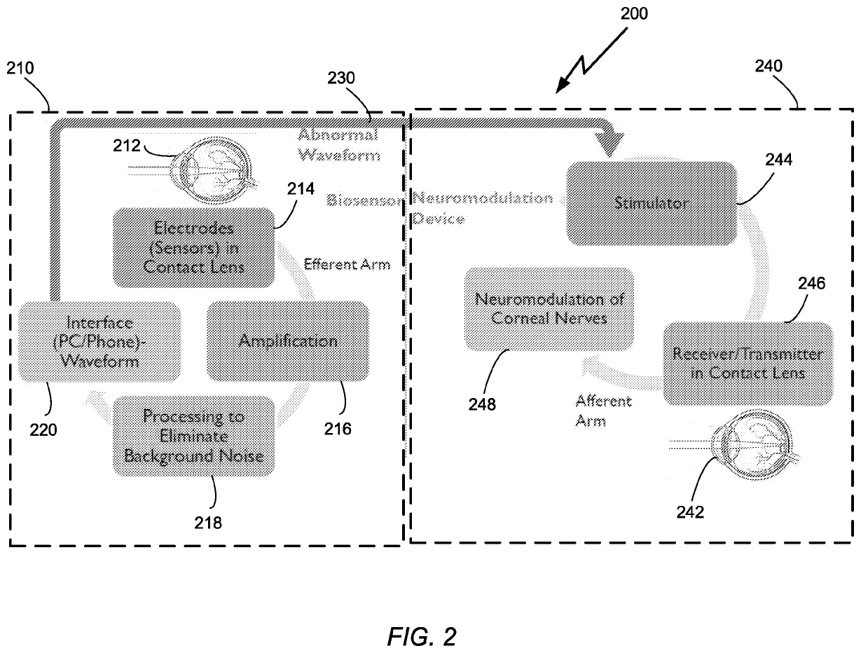

With reference next to FIG. 2, a block diagram is provided that illustrates a system 200, realizing a feedback model, which may similar to the system implementation 100 of FIG. 1, used for sensing electrical activity by nerve tissue and controllably applying stimulation (electrical, chemical, mechanical, thermal, optical, magnetic, etc.) in response to the sensed electrical activity. The model 200 includes a biosensor portion 210, configured to sense and optionally perform at least some signal processing on electrical activity produced by nerve tissue (and optionally sense other physiological features associated with the patient), and a neuromodulator device 240 that is configured to cause one or more stimulator to apply stimuli that trigger a response in a body of the patient. In some embodiments, the neuromodulation operations may be realized, at least in part, using the biosensor portion 210. The system of FIG. 2 may be implemented as a dedicated customized system, that is optimally realized (e.g., as an integral system that may be developed by a single developer) to measure electrical activity by nerve tissue and to controllably apply stimulation based on the measured electrical activity. Alternatively, the system 200 may be realized as a combination of discrete devices, unit, modules, and interfaces that are adapted/modified to operate in unison to achieve the technical solutions and objectives described herein. For example, a contact lens with electrodes, manufactured or developed by a particular manufacturer, may be combined with a remote computing system manufactured by another manufacturer and adapted to communicate with the contact lens (e.g., via wireless technologies such as Bluetooth®) to process signals measured by the electrodes on the contact lens and or to provide control signals to generate stimulation signals. Generally, any combination of commercially available units, modules, interfaces, and devices can be combined and configured to operate in accordance with the model discussed herein with respect to FIG. 2, or to implement any of the systems and methods described herein.

The example implementation of FIG. 2 may include two contact lenses 212 and 242, each of which may be similar to the contact lens 110 described in relation to FIG. 1. The neuromodulator device and the biosensor portion may be implemented on separate lenses, i.e., one lens may include, or be associated with, the biosensor portion of the system, while the other lens may include, or be associated with, the neuromodulator device. Alternatively, both lenses 212 and 242 may each implement both biosensing and neuromodulating functionality that can operate on different nerve tissue. In such examples, the system/apparatus 200 may include a first contact lens couplable to at least one first sensor and at least one first stimulator, and a second contact lens couplable to at least one second sensor and at least one second stimulator. In some such embodiments, each of the first contact lens and the second contact lens may be configured to alternately sense electrical activity of a respective at least one nerve and to stimulate respective tissue.

The contact lenses 212 and 242 may be manufactured as ‘smart lenses’ with a power source, and may also be a hard lens, a soft lens, or a hybrid lenses with a hard center comprising circuitry (e.g., realized as an integrated circuit) and a soft skirt for ease of use. The contact lens 242, for example, may include a stimulator chip constituting a neuromodulation device. Sensors 214, which may be similar to any of the sensors 130a-e described in relation to the system 100 of FIG. 1, are couplable (i.e., either in direct physical connect, or in communication with, for example, communication circuitry included in the lens) to the lens 212, and are configured to sense electrical activity produced by observed one or more nerves (e.g., corneal nerves, where the biosensor can implement an electrocorneogram) via the efferent arm of the nerve tissue. The sensors 214 (which in FIG. 2 may include electrodes embedded in the lens 212 or disposed on one of the surfaces of lens 212) detect electrophysiological output, and provide that output as electrical signals. These electrical signal may be amplified using the amplification unit 216 and processed (e.g., in a unit 218 of the biosensor 210) to decrease ‘background noise’ from tissues other than the target nerves that are being observed (e.g., in this example, to reduce noise corresponding to electrical activity originating from non-corneal nerves). For example, the signals may be processed by a software-based or hardware-based (or a hybrid combination thereof) filter implementation to remove or attenuate noise in particular bands not typically associated with electrical activity from corneal nerves. The processing and filtering operations may also include transformation to appropriate domains (e.g., frequency domain transformation) where identification of noise features may be easier. Identification of particular signal features constituting noise may also be based on signal pattern recognition that can be learned using a learning engine (e.g., a pre-trained engine implementing a neural net). The amplification and processing of electrical activity signals may be performed, in some embodiments, in circuitry housed in the electrodes or in the circuitry of the lens dedicated to performing the biosensor operations. The amplification and filtering units may be implemented as a joint single module to perform both these operations (as well as other operations). In some embodiments, at least some of the amplification and/or filtering operation may be performed at a remote processing device (such as either of the processing devices 150a and 150b of FIG. 1).

As further shown in FIG. 2, the implementation of the biosensor portion 210 of the system 200 may include a communication module 220 (a wired or wireless communication interface, which may be a transmitter or a transceiver) to communicate data representative of the non-processed (e.g., not-amplified and/or non-filtered) or partially processed (e.g., amplified and noise filtered) electrical activity signals detected through the electrodes of the biosensor 210. As noted, the biosensor, in this example, includes electrodes within or disposed on the contact lens, but the biosensor may include additional sensor located remotely from the lens, such as further remote electrodes to sense electrical activity of nerves that are farther away from the eye, as well as other types of sensors (e.g., to measure intraocular pressure, oxygen level, pulse, etc.)

Output 230 of biosensor portion 210 is communicated (through a wired or wireless link) to the neuromodulator device 240. The neuromodulator device 240 is configured to determine if the output provided by the biosensor 210 is abnormal. This processing may be performed at a stimulator 244, which may be included with the circuitry of the lens 242, or may be implemented at a remote processing device (such as either of the devices 150a or 150b). Where the stimulator functionality is implemented at a device located remotely from the lens 242, that device receives the data representative of the waveforms either directly from the biosensor device 210 (e.g., from the communication circuitry on the lens 212) or from communication circuitry on the lens 242 (which can receive that data from the lens 212).

As discussed in relation to the system 100 of FIG. 1, determination of whether an electrical activity waveform is abnormal may be performed by comparing the received waveform (which may have been amplified and filtered by the biosensor portion 210, by a remote device, or by the stimulator 244) to a repository of baseline waveforms including normal waveforms recorded from healthy individuals (which may include the particular patient being treated) for the particular nerve observed, waveforms produced when individuals were subjected to various stimuli, waveforms produced for individuals suffering from various diseases or conditions (e.g., dry eye condition, and/or any of the other conditions/diseases described herein). The comparison can be performed by aligning and/or normalizing the received waveforms to the pre-stored waveforms, and comparing statistical features of the waveform (e.g., average amplitude), general shape, frequency, and other characteristics of the measured waveform, to the corresponding features of the baseline waveforms. In some examples, comparing a received waveform to baseline waveforms may be performed by comparing individual samples of the waveforms (after performing a sampling of the received waveforms and of the baseline waveforms). For example, in comparing individual samples (typically following a normalization of the waveform), the difference between each sample of the received waveform and respective samples of a pre-stored waveform being compared to is computed. The computed difference for any two samples (or the aggregated sum of the differences of multiple compared samples) may be compared to a threshold(s). A received waveform may be deemed to be abnormal if, for example, the computed differences for samples of the waveform to one of the pre-stored waveform exceeds the corresponding threshold. In another example, a process to determine abnormal waveforms may be performed with a learning engine (implemented using a neural network procedure, a k-nearest neighbor procedure, a decision tree procedure, a random forest procedure, an artificial neural network procedure, a tensor density procedure, a hidden Markov model procedure, etc.) trained to recognize abnormal waveforms.

If the received waveform is determined to be abnormal or irregular, an output signal is produced. The output signal may be a control signal to cause the stimulation-producing devices, such as electrodes, to produce the stimulation output, or the output signal(s) may be the actual stimulation output. As discussed above, the output of the stimulator is configured to correct abnormalities by creating waveforms that, when combined with the abnormal waveform, result in a resultant waveform in which the abnormality has been lessened (or even eliminated). Alternatively, control or stimulation signals may be generated that trigger the nerves producing the abnormal waveforms to generate modified waveforms for which the abnormality with the pre-stored waveform(s) is reduced or impeded (thus causing in a correction or remedying of the abnormality). The stimulation being triggered does not need to be an electrical stimulation, but may include chemical stimulation, mechanical stimulation, thermal stimulation, optical stimulation, etc.

In some embodiments, the stimulator 234 may include a power source that generates the actual stimulation signals (e.g., when the stimulation is electrical stimulation to be applied to one or more nerves). Where the stimulator is a separate device, the power source may then send the signal in infrared waves (or via radio wave transmissions, or other types of power transfer means), that may be picked up (in the case of optical transmissions such as infrared waves) by a diode in the circuitry (chip) of the lens 242 (the diode may be part of a power module implemented on the circuitry of the lens 242). Then, the signal received at the lens 242 (via a communication/receiver module 246) may be converted to electrical output, to be delivered to the nerve tissue using electrodes (disposed or embedded on the lens, or located remotely from the lens 242, and communicatively connected to the circuitry of the lens via wired or wireless links) A neuromodulator unit 248 depicted in FIG. 2 may be configured to interface with the patient's tissue (be it nerve tissue or other tissue parts or organs). The neuromodulator unit 248 may include one or more different types of stimulator devices. The stimulator devices of the neuromodulator 248 may be electrodes that direct electrical stimulation to corneal nerves.

Electrical stimulation signals applied to the patient's tissue may be in form of biphasic, pulsed, symmetrical and charge balanced waveform with frequency between, for example, 20 and 80 Hz with voltage controlled amplitude between 5 and 50V or current controlled amplitude between 1 to 30 mA. The signal may last a period of 3-5 minutes to modulate the output from the cornea. In some embodiments, the stimulation signals (and thus the entire feedback mechanism implementations) may be maintained for a longer period of time, that could be hours, days, or longer (e.g., a patient suffering from chronic pain may need to be treated with the system 200 possibly indefinitely). Power needed by the circuitry of the lens 242 (and/or by the lens 212) may be transferred through various types of wireless power transfers. For example, power harvesting based on inductive wireless power transfers may be implemented at one or more of the lenses' circuitries. An advantage of using inductive power transfer is the source of energy does not need to be within the line of sight of the power module on the lens (in contrast, for a visible optical radiation or infrared power transfer, the power unit on the lens(es) needs to be visually aligned with the external power supply). Where inductive power transfer is used, the circuitry of the lens 242 may also include a controller unit to perform the processing of determining waveform abnormalities and generating responsive signals (e.g., implementing at least some of the functions performed by the stimulator unit 244). As noted, powering of the lenses 212 and 242, as well as the sensors and stimulators associated therewith, may also be realized through disposable or rechargeable batteries housed in the lenses, by capacitor arrays, by a mountable power source mechanism, etc.

The waveforms (the sensed output from the biosensor portion 210 and/or the stimulation signal waveforms) may be recorded in the memory of power source and the output may be sent to the user on a screen of a user interface. This user interface may send the data to the user's phone (alternatively, the user interface may be implemented as an application running on the user's phone). In some embodiments, at least the biosensor portion (e.g., one implementing an electrocorneogram, or ECG, device) may be semi-automatic and be configured to collect input at some pre-determined intervals (e.g., every 4 hours). With that semi-automatic configuration, stimulation output may be generated and applied (by the neuromodulation device) whenever an abnormal signal is detected. A treatment cycle may be executed at some pre-determined interval (e.g., at least once in every 48 hours). Alternatively, the system 200 may be configured to operate in automatic mode (where the system switches on automatically in response to some event or condition detected), or manual mode (the system switches on, and continues to operate, at the discretion of the user/patient).

In some embodiments, the system 200 (and likewise the system 100 of FIG. 1) may include one or more safety mechanisms. These mechanisms may be present at the level of stimulator, power source or user interface controls. The safety mechanisms may include implementations (e.g., circuit-based implementations) to limit the voltage, current, frequency, and duration of the stimulus when the stimulus is electrical. Voltage may be regulated using voltage regulator or boost regulator. To regulate current, transistors or resistors in series may be used. A software implementation (or alternatively, a hardware or hybrid implementation) used for the user interface may be able to set limits on the frequency and time period of the stimulation. This software may also be able to regulate the stimulator directly for this purpose.

As noted, in some embodiments, the biosensor portion and neuromodulation device may be combined so as to be included in a single lens. Thus, in such embodiments, the system 200, implemented as a single device on a single lens, is configured to not only measure irregularities in nerve function, but can modulate the nerves substantially concomitantly to correct or otherwise regulate the nerve function. For example, the combined device can determine changes (e.g., relative to baseline waveforms) in frequency, shape, and amplitude of the waveform, and generate a signal to correct those abnormalities by either a superimposition (addition or subtraction to the waveform) or by resetting the nerve discharge.

The implementations of the system 200 (and likewise the system 100) can thus improve on conventional treatment of symptoms (determined through subjective questionnaires such as the Ocular Surface Disease Index and Ocular Pain Assessment Survey (OPAS)). The implementations of the system 200 result in significant improvements in symptoms and condition indicators, such as a decrease in neuromas on confocal microscopy. Improved ocular health, including improved tolerability for contact lenses may also be seen. Additionally, some of the implementations of the system 200 can also facilitate “maintenance of systems,” e.g., nerve abnormality could be assessed prior to onset of symptoms, and thus stimulation could be done preemptively (i.e., stimulate to ensure patient does not experience symptoms). The system 200 can further be translated for use in improvement/establishment of diagnosis, relate signs to symptom severity, assess prognosis and response to therapy in various diseases and conditions. Additionally, association between symptom severity and clinical signs may be made and utilized in patient care. Furthermore, using neuromodulation as a treatment modality, new treatment regimens may be developed for corneal pain associated with various diseases.

In some examples, the stimulation applied to the patient's body to trigger a response is configured not necessarily to correct an abnormal electrical activity signal, but to achieve other therapeutic effects. For example, the stimulation applied (be it electrical stimulation, chemical stimulation, mechanical stimulation, optical stimulation, etc.), may be configured to perform one or more of, for example, promote tissue growth, promote blood vessel growth, and/or trigger an immune system of the patient to counter a medical condition (e.g., detected based, at least in part, on the sensed electrical activity produced by nerve tissue).

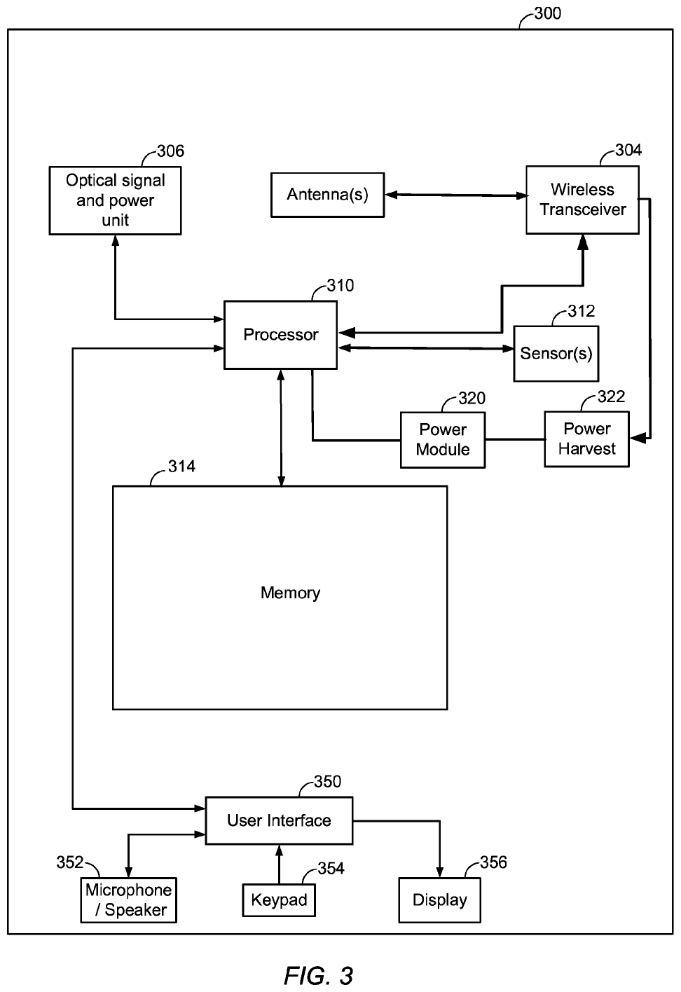

With reference now to FIG. 3, a schematic diagram of an example circuit/device 300, which may be used to implement, at least in part, the various devices, components, and circuitries depicted in FIGS. 1 and 2 is shown. For example, the example circuit 300 may be used to implement, at least partly, the circuitry 120 of FIG. 1. In another example, the circuit 300 may be used to implement, at least in part, the devices 150a or 150b of FIG. 1. It is to be noted that one or more of the modules and/or functions illustrated in the example of FIG. 3 may be further subdivided, or two or more of the modules or functions illustrated in FIG. 3 may be combined. Additionally, one or more of the modules or functions illustrated in FIG. 3 may be excluded.

As shown, the example device 300 may include a wireless transceiver 304 that may be connected to one or more antennas 302. The transceiver 304 may comprise suitable devices, hardware, and/or software for communicating with and/or detecting signals to/from a network or remote devices, and/or directly with other wireless devices within a network. In some embodiments, the transceiver 304 may support wireless LAN communication technologies (e.g., WLAN, such as WiFi-based communications), or wireless wide area network (WWAN) communication technologies (e.g., LTE, 5G, etc.) to communicate with one or more cellular access points. For example, the circuitry 120 included in contact lens 110 may be configured to communicate with a WLAN or WWAN supported device that perform at least some of the controller processing implemented by the system 100, including processing to determine whether an electrical activity waveform signal measured by a sensor is abnormal. In some variations, the wireless transceiver 304 may also support short range communication protocols (including such protocols as Bluetooth® (classical Bluetooth), Bluetooth-Low-Energy® (BLE) protocol, or proprietary protocols) that allow the device 300 to wirelessly communicate with near-by devices such as any remote sensors or remote stimulators used by the some of the various implementations described herein. The short range wireless communication protocols facilitate the communication of signals such as signals comprising data representative of waveforms measured by sensors, or signals corresponding to stimulation pulses or waveforms.

As further illustrated in FIG. 3, in some embodiments, the device 300 further includes am optical signal and power module 306 that is configured to receive optical signals (e.g., in the infrared range or in the visible optical range) encoded with data, and/or to also convert optical transmissions into power to either photoelectrically generate current from received optical radiation, or to generate optical radiation transmitted to a remote device, such as the lenses 110, 212, or 242 that can generate power from the received radiation.

The device 300 also includes one or more sensors 312 that may include sensors to sense electrical activity from proximate nerves, electrode stimulators to electrically stimulate proximate tissue, and various types of biosensor devices (oxygen sensor, heart monitor, intraocular pressure sensor, etc.) Additional types of sensors that may be included with the device 300 include motion (inertial) sensors such as an accelerometer, a gyroscope, a magnetometer (any of these motion sensors may be implemented using Micro-Electro-Mechanical Systems, or MEMS, technology), an altimeter, a thermometer (e.g., a thermistor), an audio sensor (e.g., a microphone), a camera or some other type of optical sensor (e.g., a charge-couple device (CCD)-type camera, a CMOS-based image sensor, etc., which may produce still or moving images that may be displayed on a user interface device, and that may be further used to determine an ambient level of illumination and/or information related to colors and existence and levels of UV and/or infra-red illumination), and/or other types of sensors.

The device 300 additionally includes a controller 310, which may be implemented using one or more microprocessors, microcontrollers, and/or digital signal processors, and customized control circuitry (e.g., implemented as application-specific-integrated-circuits, or ASIC) that provide processing functions, as well as other computations and control functionality. The controller 310 may also include memory 314 for storing data and software instructions for executing programmed functionality within the device. The functionality implemented via software may depend on the particular device at which the memory 314 is housed, and the particular configuration of the device and/or the devices with which it is to communicate. For example, if the device 300 is used to implement a lens with circuitry to perform waveform analysis and determination of stimulation signals, the device 300 may be configured (via software modules/applications provided on the memory 314) to implement a process to communicate with sensors and stimulators comprising the feedback system used to sense and stimulate tissue (such as nerve tissue), determine whether sensed signals (e.g., electrical activity waveforms, or signals from other types of biosensor devices) are abnormal, and generate modulating control signals, or actual stimulation signals responsive to whether, and to what extent, received signals are abnormal (relative to baseline waveforms that may be stored locally at the memory 314). The memory 314 may be on-board the processor 310 (e.g., within the same IC package), and/or the memory may be external memory to the processor and functionally coupled over a data bus.

With continued reference to FIG. 3, the device 300 may include a power module 320 such as a battery, one or more capacitors, and/or a power conversion module that receives and regulates power from an outside source (e.g., AC power). As noted, in some embodiments, the power source 320 may be connected to a power harvest unit 322. The power harvest unit 322 may be configured to receive RF communications, and harvest the energy of the received electromagnetic transmissions. An RF harvest unit generally includes an RF transducer circuit to receive RF transmissions, coupled to an RF-to-DC conversion circuit (e.g., an RF-to-DC rectifier). Resultant DC current may be further conditioned (e.g., through further filtering and/or down-conversion operation to a lower voltage level), and provided to a storage device realized, for example, on the power module 320 (e.g., capacitor(s), a battery, etc.) The power module 320 may also store energy harvested through the optical signal and power unit 306.

In some embodiments, the example device 300 may further include a user interface 350 which provides any suitable interface systems, such as a microphone/speaker 352, keypad 354, and display 356 that allows user interaction with the mobile device 300. A user interface, be it an audiovisual interface (e.g., a display and speakers) of a mobile device (such as the devices 150a of FIG. 1), or some other type of interface (visual-only, audio-only, tactile, etc.), are configured to provide status data, alert data, measured or sensed data (such as waveform data), and so on, to a user using the particular device 300. The microphone/speaker 352 provides for voice communication functionality, the keypad 354 includes suitable buttons for user input, the display 356 includes any suitable display, such as, for example, a backlit LCD display, and may further include a touch screen display for additional user input modes. The microphone/speaker 352 may also include or be coupled to a speech synthesizer (e.g., a text-to-speech module) that can convert text data to audio speech so that the user can receive audio notifications. Such a speech synthesizer may be a separate module, or may be integrally coupled to the microphone/speaker 352 or to the controller 310 of the device of FIG. 3.

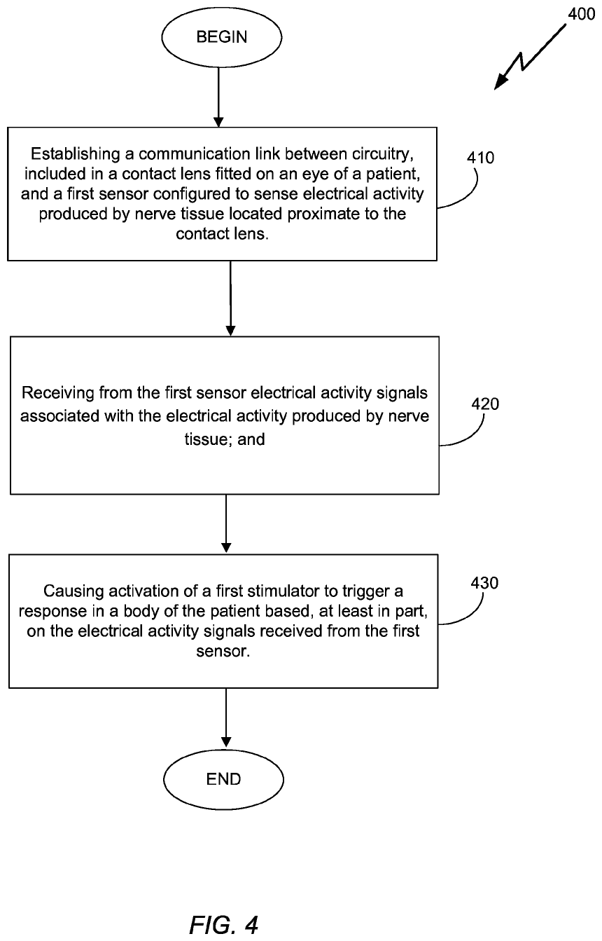

With reference next to FIG. 4, a flowchart of an example procedure 400 to sense electrical activity of nerves, and produce stimulation responsive thereto, is shown. The procedure 400 may be implemented at circuitry included in a contact lens (such as the contact lenses 110, 212, and 242 depicted in FIGS. 1 and 2) that serves as a platform through which the sensing and stimulation functionality of the procedure is implemented. Other than serving as a platform for performing the functions and operations of the procedure 400, the contact lens may also have vision correcting utility.

The procedure 400 includes establishing 410 a communication link between the circuitry, included in the contact lens fitted on an eye of a patient, and a first sensor configured to sense electrical activity produced by nerve tissue located proximate to the contact lens. As discussed herein, the first sensor (which may be an electrode-type sensor, or some other sensor device) may be included with the contact lens, in which case the communication link may be wired-based. For sensors that are located remotely from the contact lens, the established communication link may be a wireless link.

The procedure 400 further includes receiving 420 from the first sensor electrical activity signals associated with the electrical activity produced by nerve tissue, and causing 430 activation of a first stimulator (which may be an electrode-type stimulator, a chemical stimulator, a mechanical stimulator, a thermal stimulator, an optical stimulator, etc.) to trigger a response in a body of the patient based, at least in part, on the electrical activity signals received from the first sensor. Thus, the procedure 400 implements, through activation of the first stimulator (to trigger a response) based on the electrical activity signals, a biofeedback loop. In some embodiments, causing activation of the first stimulator to trigger the response in the body of the patient may include triggering electrical stimulation directed at one or more nerves in the body of the patient in response to a determination that the sensed electrical activity is abnormal.

In some embodiments, the procedure 400 may include determining whether the electrical activity signals are abnormal, and in response to a determination that the electrical activity signals are abnormal, generating modulating control signals to modulate electrical stimulation signals producible by the first stimulator. Such generated electrical stimulation signals may be applied to one or more tissue areas of the patient to reduce or impede abnormal electrical activity behavior produced by the nerve tissue. In some examples, the electrical activity signals may be representative of measured electrical activity waveforms generated due to nerve firing by at least one nerve. In such embodiments, determining whether the electrical activity signals are abnormal may include comparing the measured electrical activity waveforms to pre-stored baseline data representative of electrical activity waveforms, and generating the modulating control signals that cause the first stimulator to generate modulating electrical stimulation signals applied to the one or more tissue areas to cause the at least one nerve or related parts of the at least one nerve to vary resultant electrical activity waveforms such that differences between the resultant electrical activity waveforms and at least one baseline waveform is reduced. The pre-stored baseline data representative of the electrical activity waveforms may include one or more of, for example, a normal electrical activity waveform for a particular nerve, and a disease-caused electrical activity waveform for the particular nerve when a person is suffering from a particular irregular medical condition. In some further variations, generating the modulating control signals may include continually varying the generated modulating control signals responsive to variations in the measured electrical activity waveforms resulting from earlier modulating control signals (e.g., to define an iterative process that seeks to continually reduce, impede, or eliminate an abnormal electrical activity signal).

In some implementations, the procedure 400 may further include determining a medical condition that the patient is suffering from based on the sensed electrical activity produced by the nerve tissue. In such embodiments, the procedure 400 may additionally include determining one or more of, for example, severity of the medical condition, and/or treatment and prognosis of the medical condition. In some embodiments, the procedure 400 may further include generating storable electrical energy from wireless transmissions received by a power unit included with the circuitry of the contact lens.

The apparatus, systems, devices, and methods described herein may be used to treat (e.g., alleviate pain or discomfort) and/or identify many types of medical conditions and diseases. The following is a non-exhaustive, non-limiting list of example conditions and diseases that may be analyzed, treated, and/or mitigated with the foregoing implementations.

Several diseases of the cornea are directly or indirectly involved in stimulation of pain pathways in the eye. These include dry eye disease (DED), neuropathic corneal pain (NCP) and herpetic keratitis, as well as systemic diseases affecting corneal nerves, such as diabetic neuropathy. They not only cause changes in the nociceptors and nerve fibers of the cornea, resulting in functional alterations of the corneal nerves, but also induce plasticity by changing the central response to pain. DED is estimated to have a prevalence of up to 30%, with an estimated annual economic burden of $55.4 billion in indirect costs. According to TFOS DEWS II Definition and Classification Subcommittee, dry eye is defined as ‘a multifactorial disease of the ocular surface characterized by a loss of homeostasis of the tear film, and accompanied by ocular symptoms, in which tear film instability and hyperosmolarity, ocular surface inflammation and damage, and neurosensory abnormalities play etiological roles.’ DED is related to a decrease in tear production or quality. This disease presents with corneal pain and/or discomfort that may be described as grittiness, burning or itching. Cold thermoreceptors have been implicated in production of ocular dryness sensation and tear production; the sensation of ocular dryness in DED is caused by a change in the firing pattern of cold thermoreceptors. Anatomical abnormalities of nerves in subbasal plexus of patients with DED, studied using in vivo confocal microscopy, include a decrease in number, density and length of nerves, irregular branching patterns and an increase in the tortuosity, width, reflectivity and beading of nerves. The symptoms of DED can be quantified by several questionnaires including the Ocular surface Disease Index (OSDI), McMonnies dry eye questionnaire, Standardized Patient Evaluation for Eye Dryness questionnaire, Symptom Assessment in Dry Eye questionnaire (SANDE), National Eye Institute Vision Function Questionnaire and the Wong-Baker FACES Pain Rating Scale. However, only a handful of questionnaires including the Ocular Pain Assessment Survey (OPAS) and Eye Sensation Scale quantify ocular pain specifically. However, all the questionnaires rely on patient responses and hence are subjective measures, at best.

Neuropathic Corneal Pain, or NCP, presents with an overlap of symptoms with several conditions including DED. Etiologies implicated in this process include post-cataract surgery, post-LASIK surgery, psychiatric disease, autoimmune diseases, etc. This disease is characterized by a change in the firing patterns and threshold of nerve receptors and is characterized by allodynia (inappropriate response to nociceptive stimuli), hyperalgesia, dysesthesia and spontaneous pain. In some patients, chronic DED patients progress to develop NCP and have overlapping symptoms. While in DED the cause of symptoms is dryness or increased evaporation, in NCP, the source of the symptoms is dysfunctional nerves. However, there is no means to assess whether the pain is such patients is due to dry eye or is neuropathic in origin. There is no gold standard criterion for diagnosis of NCP; a proparacaine challenge test may be used to roughly differentiate the peripheral symptoms from those of central origin. Confocal microscopy findings in NCP patients include decreased nerve density and nerve regeneration, presence of neuromas, increased nerve tortuosity, beading and reflectivity. However, no measure is present to quantify/assess pain or improvement in pain or other symptoms without relying on patient response.

Post-herpetic neuralgia is another condition that may be treated or diagnosed using some of the implementations described herein. Although acute keratitis is easily diagnosed and treated, keratitis caused by herpes simplex virus and herpes zoster virus is associated with recurrent episodes, latency and sequela. A subset of these patients develop post-herpetic neuralgia and demonstrate altered nerve findings on confocal imaging including loss of subbasal nerves, increased nerve reflectivity, beading and presence of micro-neuromas. The nociceptors in animal studies show changes in mechanoreceptors and polymodal receptor firing patterns in herpes simplex keratitis, while cold thermoreceptors remain unaltered. The pain can impair patient functionality.

Other ocular conditions with reported nerve/nociceptor changes associated with/without corneal pain include allergic keratitis, atopic keratoconjuctivitis, Fuch's endothelial dystrophy, contact lens use (patients sometimes develop lower tolerance to lenses over time) and open-angle glaucoma. Changes in corneal nerves/nociceptors have also been implicated in several systemic diseases, although most of them do not present with corneal pain. Examples of these systemic diseases include multiple sclerosis, migraines, diabetes mellitus, fibromyalgia, migraines, Parkinson's disease, progressive supranuclear palsy, Crohn's disease, Fabry's disease and multiple endocrine neoplasia 2B.

Unless defined otherwise, all technical and scientific terms used herein have the same meaning as commonly or conventionally understood. As used herein, the articles “a” and “an” refer to one or to more than one (i.e., to at least one) of the grammatical object of the article. By way of example, “an element” means one element or more than one element. “About” and/or “approximately” as used herein when referring to a measurable value such as an amount, a temporal duration, and the like, encompasses variations of ±20% or ±10%, ±5%, or +0.1% from the specified value, as such variations are appropriate in the context of the systems, devices, circuits, methods, and other implementations described herein. “Substantially” as used herein when referring to a measurable value such as an amount, a temporal duration, a physical attribute (such as frequency), and the like, also encompasses variations of ±20% or ±10%, ±5%, or +0.1% from the specified value, as such variations are appropriate in the context of the systems, devices, circuits, methods, and other implementations described herein.

As used herein, including in the claims, “or” as used in a list of items prefaced by “at least one of” or “one or more of” indicates a disjunctive list such that, for example, a list of “at least one of A, B, or C” means A or B or C or AB or AC or BC or ABC (i.e., A and B and C), or combinations with more than one feature (e.g., AA, AAB, ABBC, etc.). Also, as used herein, unless otherwise stated, a statement that a function or operation is “based on” an item or condition means that the function or operation is based on the stated item or condition and may be based on one or more items and/or conditions in addition to the stated item or condition.

Although particular embodiments have been disclosed herein in detail, this has been done by way of example for purposes of illustration only, and is not intended to be limiting with respect to the scope of the appended claims, which follow. Features of the disclosed embodiments can be combined, rearranged, etc., within the scope of the invention to produce more embodiments. Some other aspects, advantages, and modifications are considered to be within the scope of the claims provided below. The claims presented are representative of at least some of the embodiments and features disclosed herein. Other unclaimed embodiments and features are also contemplated.

1. An apparatus comprising:
at least one contact lens fittable on an eye of a patient, the contact lens including circuitry for receiving electrical activity signals associated with electrical activity produced by nerve tissue located proximal to the contact lens;
a first sensor configured to sense the electrical activity produced by the nerve tissue and to provide the electrical activity signals; and
a first stimulator to trigger a response in a body of the patient based, at least in part, on the electrical activity signals provided by the first sensor.

2. The apparatus of claim 1, wherein the contact lens includes the first sensor.

3. The apparatus of claim 1, wherein the first stimulator is configured to produce electrical stimulation signals directed at tissue proximate to a location of the first stimulator.

4. The apparatus of claim 3, wherein the first stimulator is configured to produce the electrical stimulation signals responsive to a determination that the electrical activity signals are abnormal, and wherein the electrical stimulation signals are configured to correct, at least in part, the electrical activity produced by the nerve tissue.

5. The apparatus of claim 4, wherein the first stimulator is configured to produce the electrical stimulation signals directed at one or more nerves in the body of the patient, including at ophthalmic nerve tissue comprising one or more of an ophthalmic nerve, branches of the ophthalmic nerve, or related parts of the ophthalmic nerve, wherein the related parts comprise cell bodies and synapses associated with nerve branch pathways.

6. (canceled)

7. The apparatus of claim 1, further comprising a controller configured to:
determine whether the electrical activity signals are abnormal; and
in response to a determination that the electrical activity signals are abnormal, generate modulating control signals to modulate electrical stimulation signals producible by the first stimulator, the generated electrical stimulation signals applied to one or more tissue areas of the patient to reduce or impede abnormal electrical activity behavior produced by the nerve tissue.

8. The apparatus of claim 7, wherein the electrical activity signals are representative of measured electrical activity waveforms generated due to nerve firing by at least one nerve;
wherein the controller configured to determine whether the electrical activity signals are abnormal is configured to compare the measured electrical activity waveforms to a pre-stored baseline data representative of electrical activity waveforms;
and wherein the controller configured to generate the modulating control signals is configured to generate the modulating control signals that cause the first stimulator to generate modulating electrical stimulation signals applied to the one or more tissue areas to cause the at least one nerve or related parts of the at least one nerve to vary resultant electrical activity waveforms such that differences between the resultant electrical activity waveforms and at least one baseline waveform is reduced or impeded.

9. The apparatus of claim 8, wherein the controller configured to generate the modulating control signals is configured to:
continually vary the generated modulating control signals responsive to variations in the measured electrical activity waveforms resulting from earlier modulating control signals.

10. The apparatus of claim 8, wherein the controller is further configured to:
determine abnormality in electrical activity waveforms associated with patient pain or discomfort resulting from one or more of: stimuli and conditions detected by thermoreceptors, stimuli and conditions detected by mechanoreceptors, or stimuli and conditions detected by polymodal and other nociceptors.

11. The apparatus of claim 7, wherein the contact lens further includes at least one of: the controller, the first sensor, and the first stimulator.

12. (canceled)

13. The apparatus of claim 1, wherein the apparatus comprises a first contact lens and a second contact lens, wherein the first contact lens is couplable to the first sensor, and wherein the second contact lens is couplable to the first stimulator.

14. The apparatus of claim 1, wherein the apparatus comprises a first contact lens couplable to at least one first sensor and at least one first stimulator, and a second contact lens couplable to at least one second sensor and at least one second stimulator, and wherein each of the first contact lens and the second contact lens is configured to alternately sense electrical activity of a respective at least one nerve and to stimulate respective tissue.

15. The apparatus of claim 1, wherein the first stimulator comprises one or more stimulators that each produces one or more of: electrical output, chemical output, mechanical output, thermal output, vibratory/tactile output, magnetic output, or optical output.

16. The apparatus of claim 1, wherein the first sensor comprises multiple sensors, and wherein at least one of the multiple sensors is configured to sense the electrical activity produced by nerve tissue, and another at least one of the multiple sensors is configured to sense at least one of: chemical stimuli produced by the patient, mechanical stimuli, thermal, magnetic stimuli, or optical stimuli.

17. The apparatus of claim 1, wherein the first sensor configured to sense electrical activity produced by nerve tissue is further configured to sense at least one of: chemical stimuli produced by the patient, mechanical stimuli, thermal stimuli, magnetic stimuli, or optical stimuli.

18. The apparatus of claim 1, wherein the at least one contact lens is further configured to correct vision attributes of the eye of the patient.

19. The apparatus of claim 1, wherein the first stimulator is further configured to perform one or more of: promote tissue growth, promote blood vessel growth, or trigger an immune system of the patient to counter a medical condition detected based, at least in part, on the sensed electrical activity produced by the nerve tissue.

20. The apparatus of claim 1, wherein the first stimulator includes an implantable device with a reservoir of chemical compound, the implantable device configured to controllably release the chemical compound in the reservoir based, at least in part, on the sensed electrical activity or other measured activity produced by the nerve tissue.

21. The apparatus of claim 1, wherein the circuitry comprises a communication module, the communication module configured to communicate with one or more of the first sensor or the first stimulator via: one or more wired connections, or one or more wireless connections.

22. The apparatus of claim 1, further comprising:
a power source comprising one or more of: a charging holding device including at least one of a battery or a capacitor, a mountable power source connectable to an external power supply, or a wireless power receiver module to generate electrical current from wireless transmissions received by the wireless power receiver module with the wireless transmissions comprising one or more of: RF transmissions, or optical radiation.

23. A method comprising:
establishing a communication link between circuitry, included in a contact lens fitted on an eye of a patient, and a first sensor configured to sense electrical activity produced by nerve tissue located proximate to the contact lens;
receiving from the first sensor electrical activity signals associated with the electrical activity produced by nerve tissue; and
causing activation of a first stimulator to trigger a response in a body of the patient based, at least in part, on the electrical activity signals received from the first sensor.

24. The method of claim 23, wherein causing activation of the first stimulator to trigger the response in the body of the patient comprises:
triggering electrical stimulation directed at one or more nerves in the body of the patient in response to a determination that the sensed electrical activity is abnormal.

25. The method of claim 23, further comprising:
determining whether the electrical activity signals are abnormal; and
in response to a determination that the electrical activity signals are abnormal, generating modulating control signals to modulate electrical stimulation signals producible by the first stimulator, the generated electrical stimulation signals applied to one or more tissue areas of the patient to reduce or impede abnormal electrical activity behavior produced by the nerve tissue.

26. The method of claim 25, wherein the electrical activity signals are representative of measured electrical activity waveforms generated due to nerve firing by at least one nerve;
wherein determining whether the electrical activity signals are abnormal comprises comparing the measured electrical activity waveforms to pre-stored baseline data representative of electrical activity waveforms;
and wherein generating the modulating control signals comprises generating the modulating control signals that cause the first stimulator to generate modulating electrical stimulation signals applied to the one or more tissue areas to cause the at least one nerve or related parts of the at least one nerve to vary resultant electrical activity waveforms such that differences between the resultant electrical activity waveforms and at least one baseline waveform is reduced or impeded.

27. The method of claim 26, wherein the pre-stored baseline data representative of the electrical activity waveforms comprise comprises one or more of: a normal electrical activity waveform for a particular nerve, or a disease-caused electrical activity waveform for the particular nerve when a person is suffering from a particular irregular medical condition.

28. The method of claim 26, wherein generating the modulating control signals comprises:
continually varying the generated modulating control signals responsive to variations in the measured electrical activity waveforms resulting from earlier modulating control signals.

29. The method of claim 23, further comprising:
determining a medical condition that the patient is suffering from based on the sensed electrical activity produced by the nerve tissue; and
determining one or more of: severity of the medical condition, or treatment and prognosis of the medical condition.

30. (canceled)

31. (canceled)

32. (canceled)

33. A device comprising:
a contact lens fittable on an eye of a patient; and
circuitry included with the contact lens to:
establish a communication link between the circuitry and a first sensor configured to sense electrical activity produced by nerve tissue located proximate to the contact lens;
receive from the first sensor electrical activity signals associated with the electrical activity produced by nerve tissue; and
cause activation of a first stimulator to trigger a response in a body of the patient based, at least in part, on the electrical activity signals received from the first sensor.

34. The device of claim 33, further comprising:
one or more of: the first sensor, or the first stimulator.
